(19) United States
(12) Patent Application Publication
(10) Pub. No.: US 2003/0129598 A1
(43) Pub. Date: Jul. 10, 2003

(54) Methods for detection of differences in nucleic acids

(76) Inventors: Qinghong Yang, Mountain View, CA (US); Weihong Christy, Mountain View, CA (US)

Correspondence Address: Pennie & Edmonds, LLP, 3300 Hillview Avenue, Palo Alto, CA, 94304, US

(21) Appl. No.: 10/038,193

(22) Filed: Oct. 23, 2001

Related U.S. Application Data
(60) Provisional application No. 60/242,840, filed on Oct. 23, 2000.

(51) Int. Cl. C12Q 1/68; C07H 21/04

(52) U.S. Cl. 435/6; 536/23.1

(57) ABSTRACT

The present invention provides methods for determining the genotype of a target nucleic acid. In the methods, a target nucleic acid is contacted with a probe polynucleotide and a reference nucleic acid under conditions in which they are capable of forming a four-way nucleic acid complex with a branch structure that is capable of migration. Detection of resolution of the four-way complex under the appropriate conditions indicates the genotype of the target nucleic acid.

### 1. CROSS REFERENCE TO RELATED APPLICATIONS

This application claims the benefit under 35 U.S.C. § 119 of copending U.S. Provisional Application No. 60/242,840, filed Oct. 23, 2000. The content of this application is incorporated herein by reference in its entirety.

### 2. FIELD OF THE INVENTION

The present invention relates to the fields of molecular biology, chemistry and nucleic acid hybridization. In certain embodiments, the present invention provides methods and compositions that are useful for detecting differences between nucleic acids.

### 3. BACKGROUND OF THE INVENTION

Breakthroughs in genetics have identified numerous traits that have been associated with diseases. Such traits could be used to accelerate the prevention or treatment of the diseases. For some diseases, a single genetic marker is sufficient to indicate a predisposition for a disease. Detection of the marker can thus indicate an individual at risk for the disease. However, for many diseases, multiple genetic markers interact to generate complex genetic traits that are associated with the diseases. For such diseases, detection of multiple genetic markers might be needed to for the treatment or prevention of the disease. Methods of rapidly and accurately detecting such genetic markers are needed to improve the treatment or prevention of diseases that can be associated with genetic markers.

Many such genetic markers are single-nucleotide polymorphisms (SNPs). In almost all cases, there are two possible alleles at each SNP position. Such SNPs are distributed throughout the genome at frequency of about 1 per 1,000 base pairs. Several hundred thousand of these SNP markers are now available in public databases. These databases should facilitate the association of genetic markers with simple and complex diseases. To do so, millions of SNP scoring assays have to be done for hundred thousand of SNPs in large populations. SNP scoring is to determine which of the two alleles an individual has for the SNP of interest. Therefore, efficient methods to rapidly score SNPs are needed to utilize such genetic markers for the mapping of disease genes and effective treatment or prevention of the diseases.

Conventional methods have been used for the scoring of SNPs and other genetic markers. These conventional methods include direct sequencing of polynucleotides and direct measurement of restriction fragment length polymorphisms. In addition, methods based on the hybridization of probes to genetic markers have been used. Such methods include oligonucleotide chips, polymerase chain reaction amplification of genetic markers and other techniques.

However, such conventional techniques often suffer from poor accuracy, high cost or low throughput. For example, direct sequencing of DNA is expensive, time-consuming and inefficient. Furthermore, current hybridization-based SNP scoring methods such as SNP-chip or micro-array often lack sufficient sensitivity and/or accuracy to detect many SNPs simultaneously with a uniform set of conditions. A polynucleotide comprising one version of an SNP is often capable of hybridizing to a polynucleotide comprising the second version of the same SNP. Although hybridization is stronger between two perfectly complementary polynucleotides, single base-pair differences are often not sufficient to detect many SNPs simultaneously with the same set of conditions required for SNP-chip or micro-array.

The need thus remains for an efficient method to detect the presence or absence of sequence differences between polynucleotide samples.

### 4. SUMMARY OF THE INVENTION

Accordingly, the present invention provides methods for detecting the genotype of any polymorphism. The methods achieve sensitivities great enough to detect any genotypic variation in a nucleic acid, even a single nucleotide polymorphism. In fact, the methods of the present invention display sufficient sensitivity to accurately identify the genotype of a double stranded nucleic acid with a mismatch at a single nucleotide polymorphism.

In one aspect, the present invention provides methods for detecting the genotype of a target nucleic acid. The target nucleic acid can have a known or unknown genotype. Typically, the target nucleic acid is immobilized on a solid substrate. If the target nucleic acid is double stranded, it can be melted to yield immobilized first and second single stranded nucleic acids. Significantly, the methods of the present invention can be used to score a polymorphism in either the first or second immobilized single stranded nucleic acids, or in both immobilized single stranded nucleic acids.

An immobilized single stranded nucleic acid, either the first or second or both, is contacted with a probe polynucleotide to yield a target partial duplex by, for example, hybridization. The probe polynucleotide is typically a single stranded polynucleotide of known sequence. The probe polynucleotide and the immobilized single stranded nucleic acid can form a partial duplex with perfect complementarity in its complementary region, or a partial duplex with one or more mismatches in its complementary region.

The target partial duplex is then contacted with a reference nucleic acid under conditions in which the nucleic acids are capable of forming a four-way complex. The reference nucleic acid is typically a double stranded nucleic acid of known sequence. A four-way complex is a macromolecular structure that comprises both nucleic acids in double stranded form. Typically, a four-way complex comprises a Holliday junction. A Holliday junction is known to those of skill in the art as the branch point in a complex of two related (often identical) double stranded nucleic acids.

The conditions under which the nucleic acids are contacted are chosen so that the four-way complex is capable of branch migration. Such conditions are known to those of skill in the art and include those under which migration of a four-way junction can proceed along the strands of the nucleic acids that comprise identical or complementary sequences. Typically, conditions are chosen such that migration will proceed to completion only if the number of mismatches in the four-way complex does not increase during migration.

Depending on the number of mismatches in the four-way complex near a polymorphism in the target nucleic acid, migration of the four-way complex can halt at or near the polymorphism. If the target partial duplex comprises no mismatches in its complementary region and the reference nucleic acid shares sequence identity with the complementary region of the target partial duplex, branch migration of the four-way complex can also proceed to completion thereby resolving two double stranded polynucleotides from the complex. In addition, if the target partial duplex comprises a mismatch in its complementary region, for example at the polymorphism, migration of the four-way complex can typically proceed to completion thereby resolving two double stranded polynucleotides from the complex. Such migration can indicate that the probe polynucleotide and the immobilized single stranded nucleic acid differ in genotype.

However, if the target partial duplex comprises no mismatches in its complementary region, and the reference nucleic acid does not share sequence identity with the complementary region of the target partial duplex, then branch migration cannot go to completion and the strands of the are not resolved. The four-way complex remains immobilized on the solid support.

In the above cases when branch migration goes to completion, one double stranded sequence can be released from the solid surface, and the other double stranded polynucleotide can remain immobilized. Detection of the released polynucleotide can indicate the genotype of the target nucleic acid. The genotype of the immobilized single stranded nucleic acid can thus be resolved with by assaying the target nucleic acid with appropriate combinations of probe polynucleotides and reference nucleic acids.

Significantly, both the first and second immobilized single stranded nucleic acid can be assayed simultaneously with the same or different reference nucleic acids. Release of the appropriate polynucleotide product or products can indicate the genotype of a polymorphism on both single stranded nucleic acids simultaneously. The methods of the present invention can thus be used, for instance, to score a single nucleotide polymorphism on both strands of a double stranded target nucleic acid.

The methods and compositions of the invention can be used in any application for which the scoring of a polymorphism is useful. Such applications include genotyping, SNP scoring, nucleic acid sequencing, and so forth. The methods and compositions of the invention provide sensitive and efficient methods to score one or more polymorphisms in a single assay.

### 5. BRIEF DESCRIPTION OF THE FIGURES

FIG. 1A illustrates an embodiment of the invention for scoring one or more polymorphisms of a target nucleic acid.

FIG. 1B illustrates an alternative embodiment of the invention for scoring one or more polymorphisms of a target nucleic acid.

### 6. BRIEF DESCRIPTION OF THE TABLE

TABLE 1 provides an illustration of a method of carrying out the present invention.

### 7. DETAILED DESCRIPTION OF THE PREFERRED EMBODIMENTS

7.1 Abbreviations

The abbreviations used throughout the specification to refer to nucleic acids comprising specific nucleobase sequences are the conventional one-letter abbreviations. Thus, when included in a nucleic acid, the naturally occurring encoding nucleobases are abbreviated as follows: adenine (A), guanine (G), cytosine (C), thymine (T) and uracil (U). Also, unless specified otherwise, nucleic acid sequences that are represented as a series of one-letter abbreviations are presented in the 5′->3′ direction.

7.2 Definitions

As used herein, the terms “nucleic acid” and “polynucleotide” are interchangeable and refer to any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages.

The terms nucleic acid, polynucleotide, and nucleotide also specifically include nucleic acids composed of bases other than the five biologically occurring bases (adenine, guanine, thymine, cytosine and uracil). For example, a polynucleotide of the invention might contain at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxymethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acidmethylester, 3-(3-amino-3-N-2-carboxypropyl) uracil, (acp3)w, and 2,6-diaminopurine.

Furthermore, a polynucleotide of the invention may comprise at least one modified sugar moiety selected from the group including but not limited to arabinose, 2-fluoroarabinose, xylulose, and hexose.

It is not intended that the present invention be limited by the source of the polynucleotide. The polynucleotide can be from a human or non-human mammal, or any other organism, or derived from any recombinant source, synthesized in vitro or by chemical synthesis. The nucleotide may be DNA, RNA, cDNA, DNA-RNA, hybrid or any mixture of the same, and may exist in a double-stranded, single-stranded or partially double-stranded form. The nucleic acids of the invention include both nucleic acids and fragments thereof, in purified or unpurified forms, including genes, chromosomes, plasmids, the genomes of biological material such as microorganisms, e.g., bacteria, yeasts, viruses, viroids, molds, fungi, plants, animals, humans, and the like.

The nucleic acid can be only a minor fraction of a complex mixture such as a biological sample. The nucleic acid can be obtained from a biological sample by procedures well known in the art.

A polynucleotide of the present invention can be derivitized or modified, for example, for the purpose of detection, by biotinylation, amine modifictaion, alkylation, or other like modification.

In some circumstances, for example where increased nuclease stability is desired, the invention can employ nucleic acids having modified internucleoside linkages. For example, methods for synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide, dimethylene-sulfoxide, dimethylene-sulfone, 2′-O-alkyl, and 2′-deoxy-2′-fluoro phosphorothioate internucleoside linkages are well known in the art (see Uhlman et al., 1990, Chem,. Rev. 90:543-584; Schneider et al. 1990, Tetrahedron Lett. 31:335, and references cited therein).

The term “oligonucleotide” refers to a relatively short, single stranded polynucleotide, usually of synthetic origin. An oligonucleotide typically comprises a sequence that is 8 to 100 nucleotides, preferably, 20 to 80 nucleotides, and more preferably, 30 to 60 nucleotides in length. Various techniques can be employed for preparing an oligonucleotide utilized in the present invention. Such an oligonucleotide can be obtained by biological synthesis or by chemical synthesis. For short sequences (up to about 100 nucleotides) chemical synthesis will frequently be more economical as compared to the biological synthesis. In addition to economy, chemical synthesis provides a convenient way of incorporating low molecular weight compounds and/or modified bases during the synthesis step. Furthermore, chemical synthesis is very flexible in the choice of length and region of the target polynucleotide binding sequence. The oligonucleotide can be synthesized by standard methods such as those used in commercial automated nucleic acid synthesizers. Chemical synthesis of DNA on a suitably modified glass or resin can result in DNA covalently attached to the surface. This may offer advantages in washing and sample handling. For longer sequences standard replication methods employed in molecular biology can be used such as the use of M13 for single stranded DNA as described by J. Messing, 1983, Methods Enzymol. 101:20-78. Other methods of oligonucleotide synthesis include phosphotriester and phosphodiester methods (Narang et al., 1979, Meth. Enzymol. 68:90) and synthesis on a support (Beaucage et al., 1981, Tetrahedron Letters 22:1859-1862) as well as phosphoramidate synthesis, Caruthers et al., 1988, Methods in Enzymol. 154:287-314, and others described in “Synthesis and Applications of DNA and RNA,” S. A. Narang, editor, Academic Press, New York, 1987, and the references contained therein.

An oligonucleotide “primer” can be employed in a chain extension reaction with a polynucleotide template such as in, for example, the amplification of a nucleic acid. The oligonucleotide primer is usually a synthetic oligonucleotide that is single stranded, containing a hybridizable sequence at or near its 3′-end that is capable of hybridizing with a defined sequence of the target or reference polynucleotide. Normally, the hybridizable sequence of the oligonucleotide primer has at least 90%, preferably 95%, most preferably 100%, complementarity to a defined sequence or primer binding site. The number of nucleotides in the hybridizable sequence of an oligonucleotide primer should be such that stringency conditions used to hybridize the oligonucleotide primer will prevent excessive random non-specific hybridization. Usually, the number of nucleotides in the hybridizable sequence of the oligonucleotide primer will be at least ten nucleotides, preferably at least 15 nucleotides and, preferably 20 to 50, nucleotides. In addition, the primer may have a sequence at its 5′-end that does not hybridize to the target or reference polynucleotides that can have 1 to 60 nucleotides, 5 to 30 nucleotides or, preferably, 8 to 30 nucleotides.

The term “sample” refers to a material suspected of containing a nucleic acid of interest. Such samples include biological fluids such as blood, serum, plasma, sputum, lymphatic fluid, semen, vaginal mucus, feces, urine, spinal fluid, and the like; biological tissue such as hair and skin; and so forth. Other samples include cell cultures and the like, plants, food, forensic samples such as paper, fabrics and scrapings, water, sewage, medicinals, etc. When necessary, the sample may be pretreated with reagents to liquefy the sample and/or release the nucleic acids from binding substances. Such pretreatments are well known in the art.

The term “amplification” as applied to nucleic acids refers to any method that results in the formation of one or more copies of a nucleic acid, where preferably the amplification is exponential. One such method for enzymatic amplification of specific sequences of DNA is known as the polymerase chain reaction (PCR), as described by Saiki, et al., 1986, Science 230:1350-54. Primers used in PCR can vary in length from about 10 to 50 or more nucleotides, and are typically selected to be at least about 15 nucleotides to ensure sufficient specificity. The double stranded fragment that is produced is called an “amplicon” and may vary in length form as few as about 30 nucleotides to 20,000 or more.

The term “chain extension” refers to the extension of a 3′-end of a polynucleotide by the addition of nucleotides or bases. Chain extension relevant to the present invention is generally template dependent, that is, the appended nucleotides are determined by the sequence of a template nucleic acid to which the extending chain is hybridized. The chain extension product sequence that is produced is complementary to the template sequence. Usually, chain extension is enzyme catalyzed, preferably, in the present invention, by a thermostable DNA polymerase, such as the enzymes derived from Thermis acquaticus (the Taq polymerase), Thermococcus litoralis, and Pyrococcus firiosis.

A “Holliday junction” is the branch point in a four-way junction in a complex of two related (often identical) nucleic acid sequences and their complementary sequences. The junction is capable of undergoing branch migration resulting in dissociation into two double stranded sequences where sequence identity and complementarity extend to the ends of the strands. Holliday junctions, their formation and branch migration are concepts familiar to those of skill in the art, and are described, for example, by Whitby et al., 1986, J. Mol. Biol. 264:878-90, and Davies and West, 1998, Current Biology 8:727-27.

“Branch migration conditions” are conditions under which migration of a four-way complex can proceed along the component polynucleotide strands. Normally in the practice of the invention, conditions are chosen such that migration will proceed only if strand exchange does not result in an increase in the number of mismatches in the complementary regions of the four-way complex, wherein a net increase in the number of base mismatches can impede branch migration, resulting in a stabilized four-way complex. Appropriate conditions can be found, for example, in Panyutin and Hsieh, 1993, J. Mol. Biol. 230:413-24. In certain applications the conditions will have to be modified due to the nature of the particular polynucleotides involved. Such modifications are readily discernible by one of skill in the art without undue experimentation.

A “stabilized” four-way complex is a junction where an increase in the number of mismatches has stalled branch migration to an extent sufficient that the stabilized four-way complex is detectable and distinguishable from the duplex DNA.

Two nucleic acid sequences are “related” when they are either (1) identical to each other, or (2) would be identical were it not for some difference in sequence that distinguishes the two nucleic acid sequences from each other. The difference can be a substitution, deletion or insertion of any single nucleotide or a series of nucleotides within a sequence. Such difference is referred to herein as the “difference between two related nucleic acid sequences.” Frequently, related nucleic acid sequences differ from each other by a single nucleotide. Related nucleic acid sequences typically contain at least 15 identical nucleotides at each end but have different lengths or have intervening sequences that differ by at least one nucleotide.

The term “mutation” refers to a change in the sequence of nucleotides of a normally conserved nucleic acid sequence resulting in the formation of a mutant as differentiated from the normal (unaltered) or wild type sequence. Mutations can generally be divided into two general classes, namely, base-pair substitutions and frame-shift mutations. The latter entail the insertion or deletion of one to several nucleotide pairs. A difference of one nucleotide can be significant as to phenotypic normality or abnormality as in the case of, for example, sickle cell anemia.

A “duplex” is a double stranded nucleic acid sequence comprising two complementary sequences annealed to one another. A “partial duplex” is a double stranded nucleic acid sequence wherein a section of one of the strands is complementary to the other strand and can anneal to form a partial duplex, but the full lengths of the strands are not complementary, resulting in a single-stranded polynucleotide tail at at least one end of the partial duplex.

The terms “hybridization,” “binding” and “annealing,” in the context of polynucleotide sequences, are used interchangeably herein. The ability of two nucleotide sequences to hybridize with each other is based on the degree of complementarity of the two nucleotide sequences, which in turn is based on the fraction of matched complementary nucleotide pairs. The more nucleotides in a given sequence that are complementary to another sequence, the more stringent the conditions can be for hybridization and the more specific will be the binding of the two sequences. Increased stringency is typically achieved by elevating the temperature, increasing the ratio of cosolvents, lowering the salt concentration, and other such methods well known in the field.

Two sequences are “complementary” when the sequence of one can bind to the sequence of the other in an anti-parallel sense wherein the 3′-end of each sequence binds to the 5′-end of the other sequence and each A, T(U), G, and C of one sequence is then aligned with a T(U), A, C, and G, respectively, of the other sequence.

A “small organic molecule” is a compound of molecular weight less than about 1500, preferably 100 to 1000, more preferably 300 to 600 such as biotin, digoxigenin, fluorescein, rhodamine and other dyes, tetracycline and other protein binding molecules, and haptens, etc. The small organic molecule can provide a means for attachment of a nucleotide sequence to a label or to a support.

7.3 Methods of Scoring One or More Polymorphisms in a Nucleic Acid

The present invention is universal and permits the scoring of any polymorphism in a target nucleic acid. The polymorphism can be any mutation within a nucleic acid sequence, e.g..a single or multiple base substitution or polymorphism, a deletion or an insertion. Significantly, the methods display sufficient sensitivity to identify a mismatch at a single nucleotide polymorphism in a double stranded target nucleic acid. The methods of the invention are rapid, convenient, and amenable to automation. They are sensitive and quantitative and ideally suited for rapid mutation pre-screening and genotyping, particularly involving the identification of single nucleotide polymorphisms (SNPs).

In general, the present invention provides methods and compositions useful for scoring a polymorphism in a target nucleic acid by determining whether a four-way complex comprising the target nucleic acid and a reference nucleic acid are capable of resolving into two duplexes under the appropriate conditions. Specific embodiments of the invention are disclosed herein to illustrate the invention and to enable one skilled in the art to practice the invention. The specific embodiments are not intended to limit the scope of the invention.

7.3.1 The Target Nucleic Acid

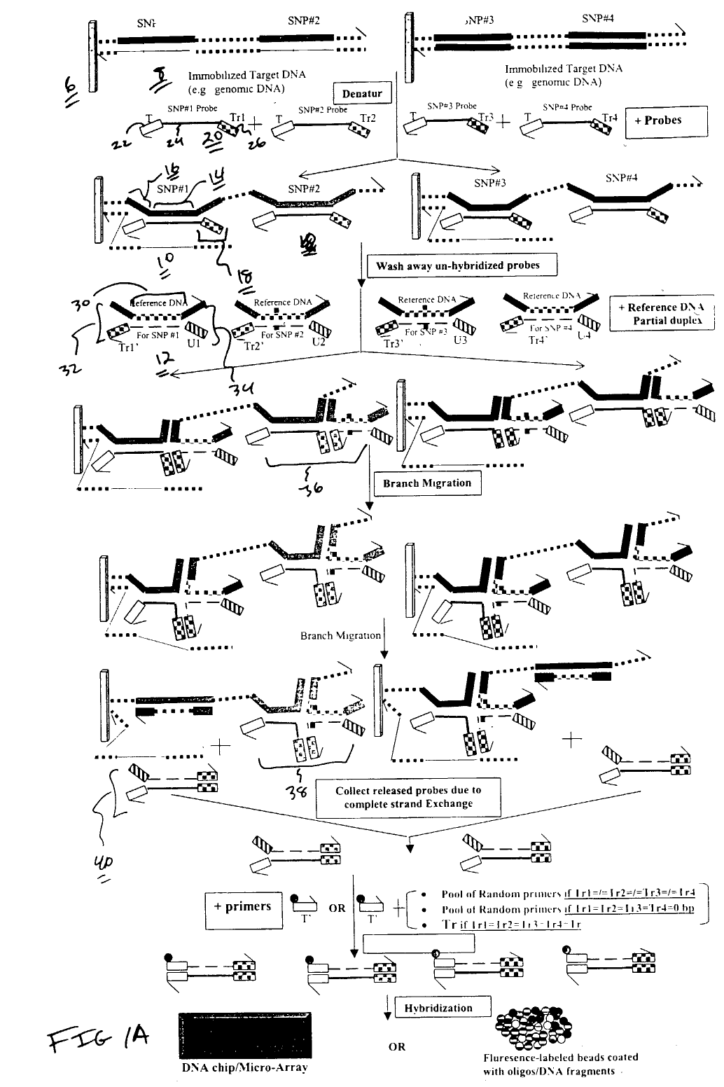

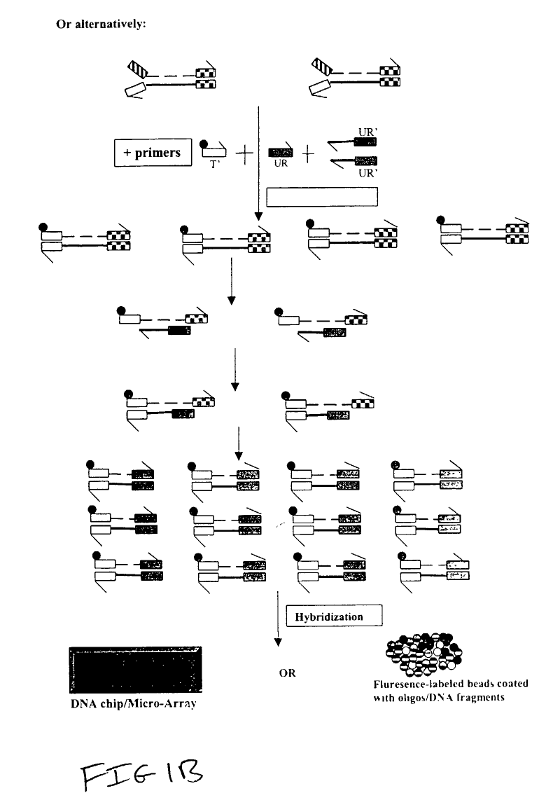

The invention provides methods and compositions for identifying the genotype of a target nucleic acid 8 by means of the formation of a four-way complex of nucleic acids comprising the sequences, as illustrated in FIG. 1A.

Typically, the target nucleic acid 8 comprises a target sequence whose genotype is to be assayed. The target nucleic acid 8 can be any nucleic acid whose sequence is to be compared to a corresponding sequence of the reference nucleic acid. The target nucleic acid 8 can be obtained from any source according to methods know to those of skill in the art. For example, the target nucleic acid can be genomic DNA, or a fragment thereof, isolated from any of the samples described in detail above.

According to the methods of the present invention, the target nucleic acid 8 is typically immobilized on a solid support 6. The solid support 6 can be any solid substrate known to those of skill in the art. The solid support 6 can comprise any material known to those of skill in the art on which a polynucleotide can be immobilized. Suitable materials include, for example, metals, polymers, glasses, polysaccharides, nitrocellulose and the like. The solid support 6 may also take on any form including beads, disks, slabs, strips or any other form capable of bearing polynucleotides. The nucleic acid can be bound to the solid support 6 by any means known to those of skill in the art for immobilizing molecules. The nucleic acid may be, for example, noncovalently associated with the solid support 6 or covalently associated directly or via a linker. In a preferred embodiment, the nucleic acid is immobilized on nitrocellulose via ultraviolet cross-linking.

For use in the methods, the immobilized target nucleic acid 8 is brought under conditions in which the polynucleotide strands of the nucleic acid are capable of separating or melting. Such conditions include any conditions known to those of skill in the art for separating the strands of a nucleic acid. For instance, the target nucleic acid 8 can be exposed to denaturing conditions such as those described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY.

In order to determine the genotype of a target nucleic acid, the target nucleic acid can be contacted with one or more probe polynucleotides. For instance, to determine the genotype of a target sequence at a single nucleotide polymorphism, the target sequence can be contacted with probe polynucleotides that complement the possible genotypes at the single nucleotide polymorphism. For instance, a first probe polynucleotide can complement the nucleobase A at a single nucleotide polymorphism, a second probe polynucleotide can complement the nucleobase G at the single nucleotide polymorphism, and so on. The probe polynucleotide that complements the genotype of the target sequence at the single nucleotide polymorphism can form a partial duplex with the target sequence having no mismatches in the complementary region. A probe polynucleotide that does not complement the genotype of the target sequence at the single nucleotide polymorphism can form a partial duplex with the target sequence having a mismatch in the complementary region at the single nucleotide polymorphism.

Thus, once denatured, a polynucleotide strand of the immobilized sample nucleic acid 8 can be contacted with a probe polynucleotide 20 under conditions in which the immobilized polynucleotide strand and probe polynucleotide 20 are capable of hybridizing to form a target partial duplex. As illustrated in FIG. 1A, a probe polynucleotide 20 comprises one or two tail polynucleotides 22 and/or 26 and a probe sequence 24 which is complementary to a sequence of the immobilized target polynucleotide.

The probe sequence 24 should complement one potential allele of a polymorphism in the immobilized target polynucleotide and surrounding sequences. In preferred embodiments of the invention, the polymorphism is a substitution, deletion or insertion variation or mutation, such as but not limited to a single nucleotide polymorphism (SNP). If the probe polynucleotide 20 complements the allele of the target sequence 8, the target partial duplex 10 formed between them will be a partial homo-duplex that has no mismatch in its complementary region. On the other hand, if the probe polynucleotide 20 does not complement the allele of the target sequence 8, the target partial duplex 10 formed between them-will be a partial hetero-duplex that has a mismatch in the duplex region.

The tail sequence 22 or 26 of the probe polynucleotide is a polynucleotide sequence which preferably displays little or no complementarity to the polynucleotide sequence of the immobilized polynucleotide. Preferably, the tail sequence 22 or 26 cannot hybridize to the immobilized polynucleotide under the contact conditions thereby allowing the probe polynucleotide 20 and immobilized polynucleotide to form a target partial duplex 10. If the probe polynucleotide 20 comprises one tail sequence, it can be at its 5′ end or at its 3′ end. The probe polynucleotide can also comprise two tail sequences at either end.

The probe polynucleotide 20 can be prepared by any method known to those of skill in the art. For instance, the probe polynucleotide 20 can be prepared by standard techniques for synthesizing oligonucleotides or according to methods of preparing tailed polynucleotides described in detail in U.S. Pat. No. 6,013,439, in U.S. Pat. No. 6,232,104 B1 and in PCT publication WO 01/69200, each of which is hereby incorporated by reference in its entirety. In preferred embodiments, the probe polynucleotide 20 is prepared by standard synthetic techniques.

A strand of the target nucleic acid 8 and the probe polynucleotide 20 should be capable of forming a target partial duplex. As illustrated in FIG. 1A, a target partial duplex comprises a complementary region 14 and one or more tail regions 16 and/or 18. In the complementary region, a portion of the probe polynucleotide 20 is capable of hybridizing to the corresponding sequence on a strand of the immobilized target nucleic acid. The complementary region of the partial duplex should comprise a significant portion probe polynucleotide 20. In a tail region, the sequence of the probe polynucleotide is not capable of hybridizing to the target sequence under typical hybridization conditions, as illustrated in FIG. 1A. The partial duplex can have a tail region at either end or at both ends. In the methods of the invention, one of the polynucleotides of the target nucleic acid is typically immobilized on a solid substrate.

7.3.2 Reference Nucleic Acid

The reference nucleic acid 12 can be a double stranded nucleic acid comprising a partial duplex of the reference sequence and its complement as illustrated in FIG. 1. The reference sequence is a sequence of the reference nucleic acid that is related to the target sequence of the target nucleic acid 8. The reference sequence is typically a known polynucleotide sequence while the target sequence is typically related to the reference sequence. The sequences can be related if the they are either identical, or would be identical if not for some difference between the two sequences. In preferred embodiments of the invention, the difference is a substitution, deletion or insertion variation or mutation, such as but not limited to a single nucleotide polymorphism (SNP). The reference nucleic acid 12, together with probe polynucleotides 20, can be used to determine the genotype of the target nucleic acid.

As the reference nucleic acid 12 comprises a partial duplex, it comprises a complementary region 30 and one or more tail regions 32 and/or 34. The complementary region 30 should comprise a substantial portion of the reference sequence. In the complementary region 30 of the reference nucleic acid, the two strands of the nucleic acid are capable of hybridizing under the appropriate conditions. In preferred embodiments, the two strands in the complementary region 30 are perfectly complementary.

A tail regioin 32 or 34 of the reference nucleic acid can be at either end of the reference nucleic acid, or tail regions 32 and 34 can be at both ends of the reference nucleic acid. In the tail region 32 or 34, the two strands of the reference nucleic acid should not be capable of hybridizing under the appropriate conditions. Preferably, the two strands of the reference nucleic acid share no significant complementarity in the tail region. Significantly, the sequence of each strand of the tail region 32 or 34 should be chosen so that the reference nucleic acid 12 is capable of forming a four-way complex with the target nucleic acid 20. Reference nucleic acids that are capable of forming a four way complex with target nucleic acids are described extensively in U.S. Pat. No. 6,013,439, in U.S. Pat. No. 6,232,104 B1 and in PCT publication WO 01/69200.

The reference nucleic acid 12 can be prepared, for example, by standard synthetic techniques or according to the tailed primer PCR methods described in U.S. Pat. No. 6,013,439, in U.S. Pat. No. 6,232,104 B1 and in PCT publication WO 01/69200, as discussed above for preparing the target partial duplex. For example, the reference nucleic acid 12 can be prepared by PCR using tailed primers from a nucleic acid comprising the reference sequence. Preferably, the two strands of the reference nucleic acid 12 are prepared by standard synthetic techniques and hybridized to form a partial duplex by standard techniques.

7.4 Determining the Genotype of the Target Nucleic Acid

The genotype of the target sequence can be detected by contacting the reference nucleic acid 12 with the partial duplex 10 under conditions in which the nucleic acids are capable of forming a four way complex 36. The four way complex 36 can be subjected to branch migration conditions.

The reference nucleic acid 12 and the partial duplex 10 can be contacted under conditions in which they are capable of forming a four way structure. Such conditions are known to those of skill in the art and can be found, for instance, in Panyutin and Hsieh, 1993, supra; in U.S. Pat. No. 6,013,439; in U.S. Pat. No. 6,232,104 B1 and in PCT publication WO 01/69200. Typically, the partial duplex 10 and reference nucleic acid 12 are brought into contact under conditions where complementary tails can anneal to one another, thereby initiating the formation of a four-stranded complex, as depicted in FIGS. 1A and 1B. The skilled artisan can determine appropriate conditions for hybridization of the tails and the resulting formation of a four-way complex of any specific duplexes. See, for example, Sambrook et al., supra., Panyutin and Hsieh, 1993, supra, and U.S. Pat. No. 6,013,439.

The resulting four-way complexes 36 are subjected to conditions where branch migration can occur. Branch migration conditions are known to those of skill in the art and can be found in U.S. Pat. No. 6,013,439, in U.S. Pat. No. 6,232,104 B1 and in PCT publication WO 01/69200. In one embodiment of the invention, branch migration is conducted in the presence of an ion such as Mg⁺⁺, which enhances the tendency of a mismatch to impede spontaneous DNA migration and hence stabilizes Holliday junction complexes involving such a mismatch. A preferred concentration range for Mg⁺⁺ is 1 to 10 mM. It should be noted that stabilization can be achieved by means of other ions, particularly divalent cations such Mn⁺⁺ or Ca⁺⁺, or by a suitable combination of ions. In a particularly preferred embodiment, branch migration is achieved by incubation at 65° C. for about 20-120 minutes in buffer containing 4mM MgCl₂, 50 mM KCl, 10 mM Tris-HCl, PH 8.3. A description of branch migration conditions suitable for the formation of stabilized Holliday junction as a consequence of a single base mismatch can be found, for example, in Panyutin and Hsieh, 1993, supra., which is hereby incorporated by reference in its entirety.

While not intending to be bound by any particular theory of operation, migration of the four-way complex 36 can proceed through a polymorphism if the four-way complex has the same number of mismatches, or fewer mismatches, after migration through the polymorphism than the four-way complex 36 had before migration through the polymorphism. For instance, if the four-way complex 36 comprises no mismatches prior to migration through the polymorphism and no mismatches subsequent to migration through the polymorphism, migration can proceed to completion thereby resolving the complex into two double-stranded polynucleotides. One of the double stranded polynucleotides 40 can be released from the solid support. However, if migration through the polymorphism forms more mismatches in the four-way complex than the complex had prior to migration through the polymorphism, migration through the complex is energetically disfavored and migration can halt at the mismatch thereby forming a stabile or immobilized four way complex 38.

Depending on the target sequence, probe sequence and reference sequence, migration of the four-way complex can halt at a mismatch, or migration of the four-way complex can proceed to completion thereby resolving the complex into two double-stranded polynucleotides. One of the two double-stranded polynucleotides can be released from the solid support.

Thus, detection and/or quantification of the released polynucleotides 40 can indicate the relationship among the target sequence, the probe sequence and the reference sequence. Detection and quantitation of the released nucleic acid 40 and/or either of its two strands under various combinations of two different probe polynucleotides and two different reference partial duplexes can be used to determine the genotype of the target nucleic acid 8.

The released duplexes 40 can be detected by any method known to those of skill in the art for detecting a nucleic acid. For instance, the released nucleic acids 40 can be detected by electrophoresis, hybridization or by other techniques known to those of skill in the art. If the reference nucleic acid 12 or target nucleic acid 10 comprises optional labels, as discussed above, then the released duplex can be detected by methods known to those of skill in the art for detecting the labels.

For the scoring of each SNP, there are many ways to combine two different probe polynucleotides with two different reference partial duplexes and then detect/quantitate the release of duplex 40 under each combination:

1) allele 1 probe polynucleotide+allele 1 reference partial duplex

2) allele 1 probe polynucleotide+allele 2 reference partial duplex

3) allele 2 probe polynucleotide+allele 1 reference partial duplex

4) allele 2 probe polynucleotide+allele 2 reference partial duplex

5) allele 1 probe polynucleotide+allele 1 and allele 2 reference partial duplex

6) allele 2 probe polynucleotide+allele 1 and allele 2 reference partial duplex

7) allele 1 and allele 2 probe polynucleotide+allele 1 reference partial duplex

8) allele 1 and allele 2 probe polynucleotide+allele 2 reference partial duplex

9) allele 1 and allele 2 probe polynucleotide+allele 1 and allele 2 reference partial duplex

For example, if a target nucleic acid has allele 1, duplex 40/probe 20 will be released under combination 1, 3, 4 but not 2. In contrast, if a target nucleic acid has allele 2, duplex 40/probe 20 will be released under combination 1, 2, 4 but not 3. Using this method to score SNPs for target nucleic acids is illustrated in more detail in Table 1. Allele 1 and allele 2 probe polynucleotides can be complementary to and hybridize with either the same strand of the denatured target nucleic acid or different strand. Moreover, under combination 7 and 8, allele 1 and allele 2 probe polynucleotides can be labeled differently so that the quantity of released allele 1 probe can be compared with that of released allele 2 probe under combination 7 and 8. If a target nucleic acid has allele 1, allele 1 and allele 2 probes will both be released due to complete strand exchange under combination 7, and wherein only allele 2 probe but not allele 1 probe will be released under combination 8.

The invention having been described, the following examples are intended to illustrate, and not limit, this invention.

### 8. EXAMPLE

Method of Determining the Genotype of a Nucleic Acid

In the following example, a method of detecting a difference between two nucleic acids is illustrated.

1. A DNA sample of interest (or target DNA, e.g.: genomic DNA or other DNA preparations that need to be genotyped) is immobilized on a solid surface. As an illustration rather than limitation, the DNA sample can be immobilized on a piece of nitrocellulose paper, baked and followed by UV cross-linking (standard procedure as in Southern blot). The target DNA can be denatured first and then immobilized on the solid surface, or it can be immobilized on the solid surface first and then denatured.

2. After the (immobilized) target DNA is denatured or while the target DNA is being denatured, a collection of probes are mixed with the immobilized and denatured target DNA. The collection of probes is comprised of n (1-10,000,000) different probes, each targeted at a specific SNP (see, FIGS. 1A and 1B, only 4 different probes targeted for 4 SNPs are shown). Each probe is comprised of three parts):

a. A 0-80 bp long (preferably 0 bp or 10-50 bp) 5′ tail Trn (Tr1, Tr2, Tr3 . . . ). When Trn=0 bp, the partial duplexes formed between the probes and their target DNA have a single tail at one end. When Trn=/=0 bp, the partial duplexes formed have double-tails at both ends. Sequences for Tin are not found in the target DNA sample and therefore will not hybridize with the target DNA. For example, for human genotyping, sequences for Tm can derive from bacteria specific sequences that have no homology with the human DNA.

b. A middle part that is unique, 1-600 bp long (preferably 5-100 bp), and contains one allele of a specific SNP. It will anneal to its target position in the target DNA sample.

c. A 3′ tail T (0-80 bp, preferably 12-50 bp)) that is universal for all probes in any one collection of probes used for each assay. Sequences for T are not found in the target DNA sample and therefore will not hybridize with the target DNA. For example, for human genotyping, sequences for Trn can derive from bacteria specific sequences that have no homology with the human DNA.

3. Wash away any probes that do not annealed to the immobilized target DNA and therefore are not bound to the nitrocellulose paper with any buffer that will allow the hybridization between the probes and the target DNA. As an illustration rather than limitation, washing buffer used for standard southern blot can be used.

4. Add a collection of n (1-10,000,000) reference DNA/partial duplexes containing one allele of each of the SNPs the probes in step 2 targeted at/hybridized with. With certain buffer ((e.g.: many commonly used buffers including TES buffer (50 mM-Tris-Hcl(PH 7.5), 50 mM NaCl, 1 mM-EDTA), TSM buffer (50 mM-Tris-Hcl(PH 7.5), 25 mM NaCl, 10 mM MgCl₂, 1 mM-EDTA), PCR buffers (with Mg++ for double-tailed partial duplexes and PCR buffers with/without Mg++ for single-tailed partial duplexes)) at certain temperature (10° C.-75° C., preferably, 37° C.-65° C.), the reference partial DNA duplexes form Holiday structures with their corresponding target partial duplexes formed (in step 2) between the immobilized target DNA and corresponding probes. The formed Holiday structures will undergo branch migration (1 minute -240 minutes, in certain buffer (e.g.: many commonly used buffers including TES buffer (50 mM-Tris-Hcl(PH 7.5), 50 mM NaCl, 1 mM-EDTA), TSM buffer (50 mM-Tris-Hcl(PH 7.5), 25 mM NaCl, 10 mM MgCl₂, 1 mM-EDTA), PCR buffers (with Mg++ for double-tailed partial duplexes and PCR buffers with/without Mg++ for single-tailed partial duplexes)) at certain temperature (10° C.-75° C., preferably, 37° C.-65° C.)).

When a target partial duplex formed (in step 2) between the immobilized target DNA and corresponding probes contains a homo-duplex allele (e.g: target DNA allele 1 anneal with probe DNA allele 1, or alternatively, target DNA allele 2 anneal with probe allele 2) that is different from the homo-duplex allele of the reference partial DNA duplex it forms a Holiday junction with, branch migration of that Holiday junction will stop and the probe will not be release from the immobilized target DNA (0 mismathces→2 mismatches, energy barrier).

On the contrary, when a partial duplex formed (in step 2) between the immobilized target DNA and corresponding probes contains a homo-duplex allele (e.g: target DNA version 1 anneal with probe DNA version 1, or alternatively, target DNA allele 2 anneal with probe allele 2) that is the same as the homoduplex allele of the reference partial DNA duplex it forms a Holiday junction with, branch migration of that Holiday junction will proceed all the way through and the probe will be release from the immobilized target DNA due to complete strand exchange (0mismatches→0 mismatches, no energy barrier).

In the case that a partial duplex formed (in step 2) between the immobilized target DNA and corresponding probes contains a hetero-duplex allele (e.g: target DNA allele 1 anneal with probe DNA allele 2, or alternatively, target DNA allele 2 anneal with probe allele 1), the Holiday junction it form with either of the two homo-duplex alleles of the reference partial DNA duplex will be resolved due to complete branch migration and the probe will be release from the immobilized target DNA due to complete strand exchange (1 mismatch→1 mismatch, no energy barrier).

Each reference partial duplex is comprised of (FIG. 1A) two strands:

a. First strand is completely complementary to the target DNA at a specific SNP position. This strand is comprised of a middle part and two sequences flanking that middle part at either the left or the right side (FIG. 1A). The middle part is the same as the middle part of its corresponding probe in step 2 that hybridizes with the target nucleic acid. The two flanking sequences are the same as the two flanking sequences of the target nucleic acid.

b. 2^{nd}/The other strand is comprised of 3 parts (FIG. 1A):

A middle part that is perfectly complementary to the middle part of the first strand.

A 0-80 bp long (preferably 0 bp or 10-50 bp) 5′ tail Un (U1, U2, U3 . . . ). Sequences for Trn are not found in the target DNA sample and therefore will not hybridize with the target DNA. For example, for human genotyping, sequences for Trn can derive from bacteria specific sequences that have no homology with the human DNA.

A 0-80 bp long (preferably 0 bp or 10-50 bp) 3′ tail Trn′ (Tr1′, Tr2′, Tr3′ . . . ) that is complementary to and can anneal with tail Trn in the corresponding probe. When Trn′=0 bp, the reference partial duplexes have a single tail at one end. When Trn=/=0 bp, the partial duplexes formed have double-tails at both ends. Sequences for Trn′ are not found in the target DNA sample and therefore will not hybridize with the target DNA. For example, for human genotyping, sequences for Trn′ can derive from bacteria specific sequences that have no homology with the human DNA.

5. Collect (and concentrate) all DNA that are not bound to the nitrocellulose after branch migration by washing with minimum amount of buffer (either the same buffer used for Holiday Junction formation and branch migration or other appropriate buffers). This collection of DNA includes excess reference partial DNA duplexes and the released probes due to complete strand exchange.

6. Any method that allows the determination of the identity of the released probes in the above collection can be used for SNP scoring. As an illustration rather than limitation:

a. amplify the probes that are released from the nitrocellulose due to complete strand exchange via using (FIG. 1A):

Labeled primer T′—which is complementary to T—and various DNA polymerases, including Taq polymerase, Taq Gold . . . , multiple cycles of DNA replication.

When Tr1=Tr2=Tr3=Trn=Tr, use primer pair labeled T′ (which is complementary to T) and unlabeled Tr to do PCR amplification.

When Trn=0 or Tr1=/=Tr2=/=Tr3=/=. . .=/=Trn, used labeled primer T′ (which is complementary to T) and a pool of random primers to do PCR amplification.

b. alternatively, selectively amplify the probes that are released from the nitrocellulose due to complete strand exchange (FIG. 1B):

universal primer T′—which is complementary to T—and various DNA polymerases, including Taq polymerase, Taq Gold . . . , multiple cycles of DNA replication (either PCR or Multiple Displacement Amplification, etc),

Universal primer UR that is not complementary to any DNA sequences in the genotyping assay,

mixture of different oligos, each oligo of the mixture is comprised of a universal 5′ tail UR′ that is complementary to UR and a 3′ portion that is part of or the whole middle portion of its corresponding probe and is therefore unique for each SNP tested

at least one of primer T′ or primer UR is labeled for detection For the identification of multiple released probes (multiplexing genotyping), the released probes selectively amplified are hybridized with DNA chip/micro-array or (fluorescent) beads that are immobilized/coated with the DNA sequences between T′ and UR for each of the n SNPs of interest.

For the identification of released probes one by one (one single SNP at a time), the presence or absence of the released oligos for a specific SNP can be detected by monitoring the amplification of the released oligos (e,g,: using fluorescent dyes such as PicoGreen or Ethidium Bromide).

7. In order to eliminate target DNA (released oligos) amplification step (by either PCR or MDA) completely, the probes can be labeled, for an example, with magnet beads and the released probes due to complete strand exchange can then be separated from reference DNA and isolated via magnet. The identification of the isolated released probes can be obtained by using hybridization with DNA chip/micro-array or (fluorescent) beads that are immobilized/coated with the n SNPs of interest.

An important issue for genotyping is accuracy. Fluctuation in the amplification step can lead to false or difficult to interpret signals. Therefore, it is important to have controls:

1. External control:

a. Genotyping a few well-studied/characterized/genotyped, (e.g: 1-10) SNPs with highly accurate but expensive genotyping assays for the target DNA and then use these SNPs as external control.

b. Mix some control DNA (with known sequences that are not found in target DNA) at comparable concentration with the target DNA before immobilization. Add control probes at comparable concentration in the probe pool before hybridization with the immobilized DNA. Also add control reference partial DNA duplexes in the reference partial DNA duplex pool. Correct or wrong scoring of these control DNA can give you a good estimation about the quality of each assay performed.

3. Internal control:

a. The collected DNA pools containing released probes from different combination of probe and reference DNA partial duplexes can be labeled differently. For example: The probes released from allele 1 probe with allele 1 reference partial DNA duplex are labeled with both label X (e.g. red label) and label Y (e.g.: green label), one label at a time. The probes released from allele 1 probe with allele 2 reference partial DNA duplex are also labeled with both label X (e.g.: red label) and label Y (e.g. green label), one at a time. The X-labeled/amplified pool of probes released from allele 1 probe with allele 1 reference partial DNA duplex are mixed with the Y-labeled/amplified pool of probes released from allele 1 probe with allele 2 reference partial DNA duplex before hybridization with the chip/micro-array. In addition, the Y-labeled/amplified pool of probes released from allele 1 probe with allele 1 reference partial DNA duplex are mixed with the X-labeied/amplified pool of probes released from allele 1 probe with allele 2 reference partial DNA duplex before hybridization with the chip/micro-array. In this case, the ratio the intensity of red signal vs. green signal will be scored instead of the absolute intensity of red or green signal. Along the same line, many different schemes of scoring can be apparent to a skilled researcher in molecular biology.

To achieve optimal accuracy, the internal controls and external controls can be combined to produce many different schemes for SNP scoring based on the method disclosed here.

Various embodiments of the invention have been described. The descriptions and examples are intended to be illustrative of the invention and not limiting. Indeed, it will be apparent to those of skill in the art that modifications may be made to the various embodiments of the invention described without departing from the spirit of the invention or scope of the appended claims set forth below.

All references cited herein are hereby incorporated by reference in their entireties.

TABLE 1
Bar code for three possible genotypes using a preferred embodiment of the
invention

| Target DNA | | | Reference | Release of | |
|---|---|---|---|---|---|
| Genotype (X/X) | Probe allele | | allele | duplex 40 | Bar code |
| 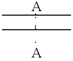 | a | 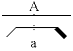 | A/A | +/+ | +/+/+/− |
| | | 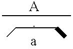 | a/a | +/+ | |
| | A | 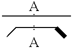 | A/A | +/+ | |
| | | 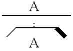 | a/a | −/− | |
| 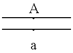 | a | 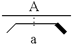 | A/A | +/− | +/+/+/+ |
| | | 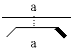 | a/a | +/+ | |
| | A | 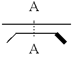 | A/A | +/+ | |
| | | 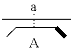 | a/a | −/+ | |
| 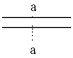 | a | 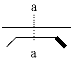 | A/A | −/− | −/+/+/+ |
| | | 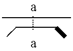 | a/a | +/+ | |
| | A | 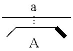 | A/A | +/+ | |
| | | 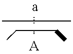 | a/a | +/+ | |

What is claimed is:

1. A method for determining the genotype of a target single-stranded polynucleotide immobilized on a solid support, said method comprising:
a. contacting the target polynucleotide with a probe polynucleotide under conditions in which the target polynucleotide and probe polynucleotide are capable of forming a target partial duplex;
b. contacting the target partial duplex with a reference nucleic acid under conditions in which the partial duplex and reference nucleic acid are capable of forming a four-way complex;
b. subjecting said four-way complex to branch migration conditions, wherein the four-way complex is capable of migrating if there is no net increase in the number of mismatches in the complex during migration, and wherein the four-way complex is capable of forming a stabile four-way complex if there is an increase in the number of mismatches in the complex during migration;
d. detecting or quantitating the release of the probe polynucleotide or release of a strand of the reference polynucleotide from the solid support as a n indication of the genotype of the target nucleic acid.

2. The method of claim 1 wherein said nucleic acid sequences are DNA.

3. The method of claim 1 wherein said four-way complex comprises a Holliday junction.

4. The method of claim 1 wherein the probe polynucleotide can be detectably labeled.

5. The method of claim 1 wherein at least one strand of the reference nucleic acid can be detectably labeled.

6. The method of claim 1 wherein release of the probe polynucleotide or release of a strand of the reference polynucleotide is detected by hybridization or electrophoresis.
